(19) United States
(12) Patent Application Publication
(10) Pub. No.: US 2012/0114772 A1
(43) Pub. Date: May 10, 2012

(54) Process For Obtaining Insoluble Substances From Genipap-Extract Precipitates, Substances From Genipap-Extract Precipitates And Their Uses

(75) Inventors: Roberta Roesler, Campinas-SP (BR); Cintia Rosa Ferrari, Sao Paulo-SP (BR); Cinthia Fernanda De Souza Ferreira, Sao Bernardo do Campo-SP (BR)

(73) Assignee: Natura Cosmeticos S.A., Itapecerica da Serra-SP (BR)

(21) Appl. No.: 13/257,697

(22) PCT Filed: Mar. 19, 2010
(86) PCT No.: PCT/BR2010/000082

§ 371 Date: Jan. 13, 2012

(30) Foreign Application Priority Data
Mar. 20, 2009 (FR) 0951813

(51) Int. Cl. A61K 36/74; A23L 1/212

(52) U.S. Cl. 424/769; 426/429; 426/615

(57) ABSTRACT

The present invention relates to the precipitation of genipap extract (Genipa americana) for obtaining a substance insoluble in polar and/or non-polar media for applications in cosmetic, pharmaceutical, textile, paint and varnish an food compositions. According to an embodiment of the invention, a process is claimed for the obtainment of substances from genipap extract precipitates comprising the steps of: a) obtaining an extract from a genipap fruit; b) adding an adsorbent substance and a mordent (precipitating agent) to the extract obtained in (a); and c) separating the solid substances precipitated in step (b). In another embodiment, the invention relates to substances from genipap extract precipitates, obtained according to the process of the invention mentioned above. The present invention further relates to a cosmetic, pharmaceutical or food composition, use and method of non-therapeutic treatment using said substances from genipap extract precipitates of the invention.

The present invention relates to the precipitation of genipap (Genipa americana) extract for obtaining insoluble substances for application in cosmetic, pharmaceutical, textile, food, paint and varnish compositions.

### BACKGROUND OF THE INVENTION

In Brazil, the plant used in the present invention is known as “jenipapo, jenipa”, among others (in English “genipap” or “genipapo”). Genipap belongs to the family Rubiacea, genus Genipa, species Americana.

The use of the genipap fruit is quite comprehensive. The ripe fruit is used more frequently in the form of liquor and sweets. In the Northeast of Brazil, genipap juice is used to fight anemia and as a diuretic against ulcers. In household medicine, the tea made from genipap roots has purgative properties, the smashed seeds induce vomit; the tea from genipap leaves has antidiarrheic effect, among other uses. The unripe fruit produces a dye that is in use since remote times by Indians and produces, by oxidation, a dye with a color going from blue to black, water-soluble, and some alcohols like ethanol and methanol. This dye is much used for dyeing fabrics, ceramics and for obtaining semipermanent tattoos.

In Brazil, the genipap plant occurs in almost every region, mainly in the Southeast, Center-West and North.

The dyeing substance is called “genipina” (genipin) and loses the dyeing effect when the fruit ripens. Thus, the more the genipap fruit is unripe the more intense the color will be. The dye, which is slightly greenish right after extraction, reacts with amino acids in the presence of oxygen, becoming blue or greenish black.

Genipin is an aglycone derived from geniposide, a glycosidic iridoid, which is found in the genipap fruit.

Iridoids are synthesized through mevalonic acid and are known technically as monoterpenoids of [c]-cyclopentane carbonic acid. The aglycones of the glycosidic iridoids have a furan or pyran in their structures.

Genipin is colorless in solution, but it reacts spontaneously with primary amino acids in the presence of oxygen and forms a pigment of bluish black color. From genipin it is possible to obtain other dyeing substances through combination or chemical substitutions.

The tanning agents derived from gallic acid, salts of aluminum salts, calcium and other polyvalent metal salts, like aluminum, iron, zinc, copper, barium, magnesium, manganese, antimony, tin, among others, are widely known in the dyeing industry and pigments as mordants for fixing dyes, acting as a precipitating agent or adsorbent, can be employed for stabilining natural dyes, the molecules of which, in the presence of light, heat and variation of pH, lose or change their color easily.

At present, due to the begin of the need to replace synthetic dyes and pigments, where many molecules have related adverse reactions, it has become important to find means to stabilize such molecules.

The inventions disclosed in patent documents PI 0005165-9, PI 0402011-1, U.S. Pat. No. 5,078,750, WO 2002/36364, US 2003/064039, US 2004/076650, US 2003/064039, JP 200-31912, JP 5-194177, JP 6-056638, JP 7-310023, JP 5-339134, JP 8-301739, JP 2001-122731, JP 8-231353, JP 200-0958859, JP 9-030949, JP 1-124322 and US 2004/0197429 show the obtainment of plant extracts, the stabilization and different application of Genipa Americana extract, mainly focusing on therapeutic uses for hair treatments, dyes for dyeing hair and drawings like semi-permanent tattoos. However, all the applications available in the prior art focus on the use of the genipap extract in the water-soluble form and do not use the Genipa americana extract in the insoluble form.

Document PI 0005165-9 relates to the obtainment of dyeing extracts and subsequent modification with tannin extract, making the plant extract dye more stable for application in the water-soluble form in cosmetic, food, paint compositions, among others.

Document PI 0402011-1 claims protection for a method of preparing a compound for drawing a non-permanent tattoo, based on the following steps: extracting a determined amount of juice from the Genipa Americana fruit (“Jenipapo”), adding a consistency agent to this juice, adding plant dyes to this mixture, among others. In this case, the compound intended for drawing is also water-soluble.

Document WO 2002/36364 discloses a system of transfer from colored sample/mold onto the skin, comprising, among its ingredients, an adhesive matrix, an adhesive polymer and at least one effective skin coloration agent, which may be a fat-dispersible organic agent from the Genipa americana extract, for example.

Document U.S. Pat. No. 5,078,750 proposes a method of dyeing/coloring hair, which comprises contacting said hair fiber with an iridoidic glycoside, which is geniposidic acid, or an aglycone, which is genipin.

Document. US 2003/064039 relates to a composition comprising, in a physiologically acceptable medium, goniochromatic pigments and additional coloration agents in the proportion of 0.1-2. Among the options of pigments, which can be part of this group of additional coloring agents are the pigments extracted from natural plant sources, as for example Genipa americana.

Document US 2004/0766650 claims protection for a composition comprising a physiologically acceptable medium, “interfering” particles that exhibit a coloring effect on the coloration of the composition, in combination with an additional dyeing agent, in an amount sufficient to mask said color imparted by the particles to the preparation. The additional coloring agent must contain in is composition at least one synthetic or organic pigment, which can be extracted from plant sources, such as (water-soluble) Genipa Americana extract. Further, it is described that the invention can be applied in hair-makeup compositions.

Document JP 200-319120 relates to a cosmetic for bath and wash, including a plant constituent, which has properties of retaining the natural skin moisturizing effect. Among the extracts mentioned as option is the huito extract (Genipa americana).

Document JP 5-194177 relates to a topical-application agent, which exhibits excellent preventive and chromatosis promoting effects, by stimulating the melanogenesis suppressing action in chromocytes. Said agent has Genipa americana extract as its active ingredient.

Document JP 6-056638 relates to an iridoidic hair dye capable of coloring the hairs in a short time and in a safe manner. Said dye can be obtained from a plant belonging to the family Rubiaceae, among others, as is the case of geniposide, geniposidic acid and genipin.

Document JP5-339134 relates to a stable hair dye, capable of dyeing the hair promptly in a specific tonality or density, without causing adverse effects in the users thereof. Said dye comprises an aglycone, which is genipin, in combination with an amino acid, like L-arginine, for coloring one's hair in a bluish black tone.

Document JP 8-301739 relates to a composition for the treatment of hair, which contains Genipa americana extract as an active dye ingredient, exhibiting an excellent uniformity with regard to the dyeing of white hair.

Document JP 2001-122731 relates to a cosmetic for bath and wash, which includes a vegetable constituent having properties of retaining the natural skin moisturizing effect. Among the extracts mentioned as option is huito extract (Genipa Americana).

Document JP 8-231353 relates to a hair tonic, which has the effect of preventing hair loss, besides stimulating the development of hair fiber. Said tonic preferably comprises huito extract (Genipa americana) as its active ingredient.

Document JP 200-095654 relates to a composition of low adhesiveness to hand skin and to hair, but with excellent capability of coloring the hair fibers. The composition is based on a blue pigment solution (genipin) extracted from gardenia, among other ingredients).

Document JP 5058859 relates to a rapid-action dye for skin and hair, based on a combination between geniposides and/or genipins and an aniline derivative.

Document US 2004/0197429 deals with the anti-inflammatory potential (inhibition of COX-2) of some plants, among which are the species belonging to the genus Genipa, like Genipa americana, for example.

### OBJECTIVES OF THE INVENTION

The present invention has the objective of obtaining insoluble substances from Genipa americana extract precipitates and using this precipitate to application, for example, in cosmetic, pharmaceutical, food, textile, paint and varnish compositions, imparting color to the human, animal or vegetable product and/or to the skin and for to the protein or cellulose material, during the administration thereof.

### SUMMARY OF THE INVENTION

This invention relates to the precipitation of substances from water-soluble genipap extract, among them the dyeing substance genipin, in its insoluble form, using mordants or adsorbent substances. The process according to the invention comprises the steps of:

a) obtaining an extract from a genipap fruit;
b) adding a mordant to the extract obtained in (a); and
c) separating the solid precipitated substances of step (b).

The present invention also relates to the precipitated solid substances obtained from a genipap extract by the above-described process, as well as to cosmetic compositions containing them and to the uses thereof.

### DETAILED DESCRIPTION OF THE INVENTION

The precipitation process of the present invention includes making a semi-purification and standardization of the genipap extract, and the mordant is preferably selected from polyvalent metal salts like, for example, aluminum, iron, zinc, copper, calcium, barium, magnesium, manganese, tin and strontium, more preferably aluminum and calcium, or from natural mordants like tannins (tanning agents), proteins, fatty acids, carbohydrates, resins, among others, more preferably compounds derived from gallic acid like tannin. In order for the desired precipitation to take place, one should take into consideration the process of purifying the extract, temperature, stirring and ranges of adequate and specific pH for each reactant used and that, therefore, are no limiting parameters of the invention.

Considering that the crystallization and precipitation process is extremely complex, non-colorant compounds present in the extract, such as amino acids, minerals, tannins, sugars, saponines, cumarins, flavonoids, etc., generally play a fundamental role in complexing it with the substrates and the mordents, in order there to be destabilization of the particle, crystallization and, finally, precipitation. Thus, it is important to define the exact degree of purification, so as to obtain a sufficiently standardized and pure precipitate for cosmetic application (without the risk of toxicity, microbial contamination, etc.), but, at the same time, with sufficient amounts of non-dyeing compounds for complexation and formation of crystals. In the case of genipap extract, the present invention preferably uses a semi-purified and standardized extract that maintains adequate levels of non-dyeing compounds, indispensable for the complexation and crystallization of the extract. Such non-dyeing compounds can vary in the extract from 5 to 20%, preferably from 10 to 16%, by weight. Additionally, for the color-standardization process one may add compounds like amino acids, in order to balance the initial amount of this compound, which varies depending on the ripening state of the fruit between 100 and 800 ppm according to the extracts obtained during the study.

The process of the invention transforms a water-soluble genipap extract into an insoluble precipitate. Soluble parts are also formed, but they can then be removed from the insoluble product by washing. Preferably, the water-soluble precipitate, when formed, should be removed almost completely.

The substances obtained by precipitating genipap extract according to the invention are insoluble in polar and/or non-polar media. The advantage of the insoluble form is that it can be used as suspensions in water-soluble and liposoluble formulations, as well as in powdered formulation (e.g. shade, blush, etc.). Additionally, the insoluble form exhibits high covering (coloration) of the skin. The water-soluble form of the genipap extract, even in powder (after drying by spray-drier or lyophilization), does not impart coloration with high skin covering.

Depending on the type of final formulation desired, a compositions according to the present invention may use either the insoluble form or the insoluble and water-soluble forms together. In order to obtain the formulation, one does not carry out solubilization, but rather dispersion. Some compositions according to the invention may use only the insoluble extract, and others may use the soluble and insoluble extracts together in order to obtain synergism. It is not possible to obtain adequate and desirable coloration and covering For make-up procedures, without the insoluble form being present.

According to a preferred embodiment, the present invention relates to a process for obtaining substances from genipap extract precipitates, comprising the steps of:
a) obtaining an extract from a genipap fruit; preferably one defines beforehand the ripening degree, considering parameters like texture, diameter and color, which will depend on the specific fruits used;
b) semi-purification of the raw extract and qualification of non-dyeing compounds;
c) standardization of the extract by adding non-dyeing compounds;
d) adding an adsorvent and a mordant to the extract obtained in (c);
e) separating the solid substances precipitated from step (d);
f) washing and adjusting the electric conductivity of the precipitates;
g) drying in an oven; and
h) grinding or sieving for homogenization of the particle size of the dried precipitate obtained in (g).

All the process is carried out under adequate and specific conditions of temperature, stirring an pH, which, however, should be determined according to each specific case and do not represent parameters limitative of the process of the present invention.

According to a preferred embodiment of the invention, in step a) the genipap fruit extract is obtained by grinding the unripe fruit with defined size and texture, respectively, between about 20 mm and about 150 mm, preferably between about 39 and about 90 mm, and between approximately 500 gf and approximately 10,000 gf, preferably approximately 1,000 gf and approximately 6,000 gf, under high rotation of about 1,000 to about 5,000 rpm, preferably about 1,800 rpm. The extract is separated from the fruit by centrifugal force, the extract being then cooled down to between 2 and approximately 10° C., preferably approximately 5° C., and filtered in a press or vacuum filter.

Then, the genipap extract is standardized by adding primary amino acid, preferably glycin (following parameters detailed in step c) mentioned before), heated up to the temperature of about 50 to about 95° C., preferably about 80° C., for a time that may vary from about 30 minutes to about 5 hours, preferably about 1 hour.

In said preferred step c), the amino acid may be selected based on the genipin content present, and primary amino acids, among which are, in varying amounts, glutamic acid, serine, glycine, hystidine, arginine, threonine, alanine, proline, tyrosine, valine, methionine, cystine, isoleucine, leucine, phenylanine and lysine, which are the chiefly responsible for the formation of coloration that will give rise to the precipitated insoluble extract.

It is possible to standardize the soluble genipap extract by adding primary amino acids like those cited above, preferably glycine, under heating up to between about 50 and about 90° C., preferably 80° C., for a time of approximately 30 minutes to approximately 5 hours, preferably approximately 1 hour, under stirring. The amount of amino acid to be added ranges from about 0.10 to about 5.00% with respect to the mass of genipap extract and will be established during the standardization for obtaining an extract with final coloration determined in reflectance spectrophotometer, the L*a*b* values of which are between: L*=10.00−50.00, preferably 24.00+/−0.30, a*=(−1.00)−3.00, preferably 0.20+/−0.50 and b*=0.10−2.00, preferably 0.20+/−0.10. The mordant used in step d) may be selected from:
(1) polyvalent metal salt, which is added as a solution or a dispersion of polyvalent metal salt, preferably the solution or the dispersion of polyvalent metal salt comprising polyvalent metals selected from the group consisting of aluminum, iron, zinc, copper, calcium, barium, magnesium, manganese, antimony, tin, strontium, among others. Still more preferably, the solution of polyvalent metal Salt comprises calcium or aluminum salts comprising CaCO3 or Al₂(SO4)₃.18H₂O.

In general, lacquers are produced by adding a substrate and a precipitating agent. Both are classified as mordants. In the case of the insoluble compound produced with genipap extract, the substrate used was calcium carbonate (adsorvent), and the precipitating agent was tannin. Tannin binds to the extract dye, and calcium carbonate adsorbs same. In some cases, other calcium salts are used with precipitating agents binding to the dye. Both the chemical linkage and the adsorption depend on ideal conditions of pH and temperatures, as described herein. The precipitating agents or the adsorbents are selected according to the affinity of each dye, and are not equal in all the processes for obtaining colored insoluble compounds.
(2) Natural substrate, which is added as a solution of natural substrate, preferably the natural substrate solution comprising tannins (tanning agents), proteins, fatty acids, carbohydrates, resins, among others. Still more preferably, the natural substrate solution comprises compounds derived from gallic acid like tannin (an aqueous solution of tannin extract).

In step d), after heating the substrate and still under heating, an aqueous suspension of calcium carbonate is added at approximately 2 to approximately 40%, preferably approximately 9%, by weight based on the total weight of the extract, followed by addition of a solution of hydrated aluminum sulfate, zinc sulfate, copper sulfate, or other polyvalent metal salts like barium, magnesium, manganese, antimony, tin and strontium salts, or other aluminum, zinc and copper salts that are not sulfates, previously acidified with an acid like hydrochloric acid, sulfuric acid and acetic acid, preferably acetic acid, until a range of pH of about 2 to about 6, preferably about 4 to about 5, is reached. The mixture of extract and calcium carbonate should be maintained under heating and stirring for about 30 minutes to about 5 hours, preferably about 1 hour, at a temperature of about 50 to about 96° C., preferably about 80° C.

Preferably, calcium carbonate is used due to its morphology and physicochemical properties. Additionally, another mordent may be used that will act in an adjuvant manner with calcium carbonate. Since calcium carbonate makes the adsorption of the extract possible, calcium carbonate together with another mordent fixes the extract, making it insoluble in polar and/or non-polar media.

After adding calcium carbonate to the extract, another mordent (natural substances or polyvalent metal salts) is added and after obtaining the precipitate in an aqueous medium, the solid substance obtained is filtered under vacuum or in press-filter, by procedures and with equipment usually employed for such purpose.

The solid substance obtained is washed to remove remaining water-soluble extract, and its conductivity is adjusted by the washes them selves or, if necessary, by adding a positive or negative electrolyte that enables one to obtain the desired conductivity between 50 mV and 150 mV, preferably between 70 and 90 mV.

The solid substance obtained, insoluble in polar or non-polar substances, is preferably dried in an oven, at a temperature of about 30 to about 80° C., more preferably of about 50 to about 60° C., until the final moisture reaches about 0 to about 10%, preferably about 1 to about 2%.

The dried solid substance is then ground until its particle size reaches a range of about 15 μm to about 150 μm, preferably about 30 to about 50 μm.

According to a preferred embodiment, the process of the present invention is carried out from natural acacia extract, which contains at least 73% of tanning agents and calcium carbonate.

Soluble substances from genipap-extract precipitate obtainable according to the process of the invention are also embodiments of the present invention, the physicochemical characteristics of which are illustrated ion Table 1 below:

TABLE 1
Physicochemical characteristics

| | |
|---|---|
| Aspect | Powder |
| Color | Blue |
| pH (10% aqueous solution) | About 4 to about 8 |
| Particle size | About 15 to about 150 μm |
| Apparent density (g/cm³) | About 0.2000-about 0.9500 |
| Total ashes | About 20-about 80% |

The present invention further relates to compositions comprising the insoluble substances from genipap extract precipitates, obtained by the process described above, and to a carrier or excipient.

The present invention also refers to cosmetic, pharmaceutical, food, textile and paint compositions, which comprise insoluble substances from genipap extract precipitates and a carrier or excipient that, in the case of cosmetic, pharmaceutical, food compositions, is a physiologically acceptable carrier or excipient. The term “physiologically acceptable” should be understood within the broadest scope available in the art, indicating, in general, that the substance in question does not interact, harm or cause side effects to the normal functions of the cells, tissues, organs and systems of healthy living beings.

Said cosmetic, pharmaceutical or food compositions may further comprise active compounds selected from the group consisting of: sunscreens, color agents, emollients, antioxidants, film-forming agents, structure agents, thickeners, wetting agents, pH-adjusting agents, preservatives, sensorial agents, suspending agents, binders, disagglomerating agents, emulsifiers or combinations thereof.

The carriers, excipients and active compounds mentioned above embrace all the substances usually employed in the cosmetic, pharmaceutical, textile, paint and varnish and food industries. By way of example, without restriction of the scope of the invention, the following are cited:
sunscreens: 2-ethylhexyl methoxycinnamate, titanium dioxide;
color agents: organic or inorganic pigments;
emollients: capric/caprylic triglyceride, stearoyl isocetyl, certified natural alpha-bisboloi FSC, dicaprylic ether, essential/plant oils (e.g. castor oil, hydrogenated castor oil); capric/caprylic glycerides, Murumuru butter, decyl oleate, polybutene, isopropyl palmitate;
antioxidants: tocoferol; ascorbyl palmitate, ascorbic acid, citric acid, butyl hydroxyl toluene;
structure agents: stearic acid, candelilla wax, carnauba wax, sorbitan olivate, ceresin, glyceryl triberrenine/berrenate;
thickeners: xanthan gum, stearic acid;
wetting agents: glycerin, plant glycerin, propylene glycol;
pH-adjusting agents: triethanolamine;
preservatives: potassium sorbate, phenoxyethanol, DMDM hydantoin;
sensorial agents: glyceryl tribehenin/behenate, mica, tapioca starch;
suspensing agent: propylene stearalconium hectorite/carbonate, coloidal silicon dioxide, aluminum and magnesium silicate;
binders: synthetic wax, glyceryl tri-behenine/behenate, stearoyl isocetyl;
disagglomerating agents: colloidal silicon dioxide;
emulsifiers: glyceryl stearate, sorbitan olivate, cetearyl olivate, sorbitan olivate;
carrier/excipients: water, plant extracts (e.g. genipap extract), talc.

The cosmetic, pharmaceutical or food compositions according to the present invention may comprise emulsions for face and body and make-up, waxy and oily products, powders, toilet soaps, (aqueous or oily) gels, (aqueous, oily or emulsion) pastes and lotions.

Another embodiment of the invention relates to the use of insoluble substances from genipap extract precipitates according to the present invention in the cosmetic, pharmaceutical, textile, paint and varnish and food industries. Particularly, the invention comprises the use of the insoluble substances from genipap extract precipitates, also an object of the present invention, in preparing cosmetic, pharmaceutical or food compositions, imparting color to the product.

Another embodiment of the present invention is a non-therapeutic method of treating humans and animals, which comprises the topical administration of the insoluble substances from genipap extract precipitates of the present inventions or the cosmetic, pharmaceutical or food compositions of the present invention to humans or animals, imparting color to the skin and/or keratinic material thereof.

### EXAMPLES

For illustrative purposes, which should not be interpreted as being limitative of the scope of the invention, some examples are presented below related to some embodiments of the inventions: (i) process for obtaining substances from genipap extract precipitates; (ii) substances from genipap extract precipitates, and (iii) cosmetic composition containing substances from genipap extract precipitates. Powders like shades, emulsions like eyelash mascaras and eyeliners, and gels using the genipap extract precipitates according to the present invention were prepared by processes and techniques usual in organic chemistry, cosmetics and pharmacy.

### Example 1

### A Process for Obtaining Substances from Genipap Extract Precipitates and Substances from Genipap Extract Precipitates

A process for obtaining substances from genipap extract precipitates were carried out by the steps of:
a) extracting the dye from the unripe genipap fruit previously classified, grinding the fruit under high rotation and separating the juice from the unripe fruit by centrifugal force; the extract was then cooled and filtered;
b) standardizing the genipap extract by adding glycin and heating the genipap extract up to a temperature of about 80° C., for about 1 hour;
c) after heating the extract in step b), one added to the extract, still under heating, an aqueous dispersion of calcium carbonate at 9%, previously acidified with acetic acid, until the pH range of about 4 to about 5 was reached; in this step the mixture of extract and calcium carbonate solution was kept under heating and stirring for about 1 hour, at a temperature of about 80° C.;
d) after adding the calcium carbonate to the extract in step c), one added to the extract a mordant comprising an aqueous solution of Al₂(SO4)₃.18H₂O:
   d.1) addition of the aqueous solution of Al₂(SO4)₃.18H₂O: one dissolved the Al₂(SO4)₃.18H₂O at 9% in water, in a pH range of about 0.9 to about 2; one added the solution of Al₂(SO4)₃.18H₂O to the mixture of genipap extract and calcium carbonate and kept under stirring and heating at about 80° C. for 1 hour;
e) after obtaining the precipitate in an aqueous medium, the solid substance obtained was filtered under vacuum or in a press-filter, by procedures and with equipment usually employed in the art for this purpose;
f) the solid substance obtained in step d) was washed with demineralized water, and its conductivity was reduced until conductivity values between 50 and 150 mV, preferably between 70 and 90 mV, were achieved;
g) the solid substance obtained, insoluble in polar or non-polar mediums, was dried in an oven, at a temperature of about 50 to about 60° C., until a final moisture of about 1 to about 2% was reached;
h) the dried solid substance was ground until its particle size reached about 15 μm to about 150 μm, preferably about 30 to about 50 μm.
   The powdered solid substances obtained from genipap extract precipitates obtained by the process described above exhibited the physicochemical characteristics described in Table 2 below:

TABLE 2
Physicochemical characteristics

| | |
|---|---|
| Aspect | Powder |
| color | blue |
| pH (10% aqueous solution) | About 4 to about 8 |
| Particle size | About 15 to about 150 μm |
| Apparent density (g/cm3) | About 0.2000 to about 0.9500 |
| Total ashes | About 20 to bout 80% |

### Example 2

### A Process for Obtaining Substances from Genipap Extract Precipitates and Substances from Genipap Extract Precipitates

A process for obtaining substances from genipap extract precipitates were carried out by the steps of:
a) extracting the dye from the previously classified genipap unripe fruit, grinding the fruit under high rotation and separating the juice from the unripe fruit by centrifugal force; the extract was then cooled and filtered;
b) standardization of the genipap extract by adding glycin and heating the genipap extract up to a temperature of about 80° C. for about 1 hour;
c) after heating the extract in step b), one added to the extract, still under heating, an aqueous dispersion of calcium carbonate at 9%, previously acidified with acetic acid, until the pH range of about 4 to about 5 was reached; in this step the mixture of extract and calcium carbonate solution was kept under heating and stirring for about 1 hour, at a temperature of about 80° C.;
a) after adding calcium carbonate to the extract in step c), one added to the extract the mordant comprising an aqueous solution of tannin extract;
   d.1) addition of the aqueous solution of tanin; a 9% tannin extract is dissolved in water at a pH range of 4 to 5 and is added to the mixture of genipap extract and calcium carbonate and kept under stirring and heating at about 80° C. for 1 hour;
e) after obtaining the precipitate in an aqueous medium, the solid substance obtained was filtered under vacuum or in press-filter, by the procedures and with the equipment usually employed in the art for this purpose;
f) the solid substance obtained in step d) was washed with demineralized water and its conductivity was reduced until a conductivity values between 50 and 150 mV, preferably 70 and 90 mV;
g) the solid substance obtained, insoluble in polar or non-polar mediums, is dried in an oven, at a temperature of about 50 to about 60° C. until a final moisture of about 1 to about 2% is reached;
h) the dried solid substance is ground until its particle size reaches about 15 μm to about 150 μm, preferably about 30—to about 50 μm.
   The substances powdered solid substances from genipap extract precipitates obtained by the process described above exhibited the physicochemical characteristics described in Table 3 below.

TABLE 3
Physicochemical characteristics

| | |
|---|---|
| Aspect | Powder |
| Color | Blue |
| pH(10% aqueous solution) | About 4 to about 8 |
| Particle size | Bout 15 to about 150 μm |
| Apparent density | About 0.2000 to about 0.9500 |
| Total ashes | About 20 to about 80% |

### Example 3

### Eyeliner

| | Concentration | Preferred | |
|---|---|---|---|
| Component | range | amount | Function |
| Genipap extract | 30-70 | 58.750 | Carrier |
| Glycerin | 1.5-6.0 | 4.000 | Wetting agent |
| Propylene glycol | 0.50-3.00 | 2,000 | Wetting agent |
| Aluminum and | 0.05-1.0 | 0.000 | Suspending |
| magnesium silicate | | | agent |
| Xanthan gum | 0.1-1.0 | 0.300 | Thickener |
| Insoluble genipap | 0.5-50.0 | 30.000 | Color agent |
| precipitate obtained | | | |
| according to example | | | |
| 1 or 2 | | | |
| Stearic acid | 0.10-1.00 | 0.500 | Structure |
| | | | agent/thickener |
| Glyceryl stearate | 0.1-0.50 | 0.100 | Emulsifier |
| Sorbitan olivate | 0.1-1.00 | 0.750 | Emulsifier |
| Cetearyl olivate, sorbitan | 1.00-5.00 | 1.000 | Emulsifier |
| olivate | | | |
| Capric/caprylic | 0.1-2.00 | 0.500 | Emollient |
| triglyceride | | | |
| Capric/caprylic | 0.50-0.80 | 0.100 | Suspending |
| triglyceride, | | | agent |
| stearalconium hectoryl, | | | |
| propylene carbonate | | | |
| triethanolamine | 0.05-1.00 | 0.100 | pH adjust |
| Potassium sorbate | 0.05-0.10 | 0.100 | Preservative |
| water | 0.50-1.00 | 1.0000 | Carrier |
| phenoxyethanol | 0.1-1.00 | 0.600 | dye |

### Example 4

### Shade

| | Concentration | Preferred | |
|---|---|---|---|
| Component | range | amount | Function |
| Potassium sorbate | 0.50-0.10 | 0.100 | Preservative |
| Synthetic wax | 0.05-1.00 | 0.100 | Binder |
| Talc | 1.00-90.00 | 35.540 | Carrier |
| Colloidal silicon dioxide | 0.005-0.010 | 0.010 | Disagglomerating |
| | | | agent |
| Dlyceryl tri- | 1.00-10.00 | 4.000 | Sensorial agent |
| behenin/behenate | | | |
| PEG-8, tocopherol, | 0.1-0.05 | 0.050 | Antioxidant |
| ascorbyl palmitate, | | | |
| ascorbic acid | | | |
| and citric acid | | | |
| Stearoyl isocetyl stearate | 0.50-10.00 | 4.000 | Emollient/binder |
| Phenyloxyethanol | 0.1-1.0 | 1.000 | Preservative |
| Certificated natural | 0.05-1.00 | 0.200 | emollient |
| alpha-bisabolol FSC | | | |
| Insoluble genipap | 1.00-90.00 | 50.000 | Color agent |
| precipitate obtained | | | |
| according to example 1 | | | |
| or 2 | | | |

### Example 5

### Cream Shade

| | Concentration | Preferred | |
|---|---|---|---|
| Component | range | amount | Function |
| PEG-8, tocopherol, | 0.05-0.10 | 0.100 | Antioxidant |
| ascorbyl palmitate, ascorbic | | | |
| acid and citric acid | | | |
| Dicaprylic ether | 1.00-20.00 | 10.000 | Emollient |
| Capric/caprylic triglyceride | 5.00-20.00 | 12.400 | Emollient |
| Castor oil | 2.00-30.00 | 23.500 | Emollient |
| Capric/caprylic glycerides | 0.10-1.00 | 0.500 | Emollient |
| Insoluble genipap | 1.00-50.00 | 20.000 | Color agent |
| precipitate obtained | | | |
| according to example 1 or 2 | | | |
| Tapioca starch | 1.00-25.00 | 20.000 | Sensorial agent |
| Murumuru butter | 1.00-10.00 | 3.000 | Emollient |
| Candelilia wax | 1.00-15.00 | 3.500 | Structure agent |
| Carnauba wax | 1.00-15.00 | 4.000 | Structure agent |
| Sorbitan olivate | 1.100-10.00 | 3.000 | Structure agent |

### Example 6

### Gel Shade

| | Concentration | | |
|---|---|---|---|
| Component | range | Preferred amount | Function |
| Xanthan gum | 0.10-2.00 | 1.0000 | Thickener |
| Plant glycerin | 0.10-5.00 | 1.0000 | Wetting agent |
| Standardized | 10.00-90.00 | 88,4009 | Color agent |
| genipap extract | | | |
| Demineralized | 0.50-90.000 | 5.0000 | carrier |
| water | | | |
| Triethanolamine | 0.10-1.00 | 0..0000 | pH adjust agent |
| Acrylate copolymer | 1.00-10.00 | 2.0000 | Film-former |
| Potassium sorbate | 0.05-2.00 | 0.10000 | Preservative |
| Demineralized | 1.00-2.00 | 1.0000 | carrier |
| water | | | |
| DMDM hydantoin | 0.50-1.00 | 1.0000 | Preservative |
| Insoluble genipap | 1.00-20.00 | 5.0000 | Color agent |
| precipitate | | | |
| obtained according | | | |
| to example 1 | | | |
| or 2 | | | |

### Example 7

### Lipstick

| | Concentration | Preferred | |
|---|---|---|---|
| Component | range | amount | Function |
| 2-ethylhexyl | 1.00-6.00 | 2.00 | Sunscreen |
| methoxycinnamate | | | |
| Titanium dioxide | 1.00-4.00 | 1.00 | Sunscreen |
| Pigment | 0.50-10.00 | 2.00 | Color agent |
| Insoluble genipap | 0.50-10.00 | 4.00 | Color agent |
| precipitate | | | |
| obtained according | | | |
| to example 1 | | | |
| or 2 | | | |
| Decyl oleate | 5.00-20.00 | 12.00 | Emollient |
| Castor oil | 20.00-70.00 | 55.15 | Emollient |
| Tuloene butyl | 0.01-0.05 | 0.05 | Antioxidant |
| hydroxy | | | |
| Glyceryl abietate | 1.00-3.50 | 2.00 | Film former |
| Carnauba wax | 0.50-5.00 | 1.00 | Structure agent |
| Candelilla wax | 0.50-7.00 | 4.00 | Structure agent |
| Ceresine | 0.050-5.00 | 3.50 | Structure agent |
| Hydrogenated | 0.50-8.00 | 3.30 | Emollient |
| castor oil | | | |
| Polybutene | 2.00-10.00 | 7.50 | Emollient |
| Isopropyl palmitate | 1.00-10.00 | 2.50 | Emollient |

1. A process for obtaining substances from genipap extract precipitates, characterized by comprising the following steps:
a) obtaining an extract from a genipap fruit;
b) adding a mordent to the extract obtained in (a); and
c) separating the solid substances precipitated from step (b).

2. A process according to claim 1, characterized in that, in step b), the mordent is selected from the group comprising polyvalent metal salts and/or natural substrate.

3. A process according to claim 2, characterized in that the polyvalent metal salt is selected from the group consisting of aluminum, iron, zinc, cop per, calcium, barium, magnesium, manganese, antimony, tin and strontium.

4. A process according to claim 3, characterized in that the polyvalent metal salt comprises calcium or aluminum salts comprising CaCO₃ or Al₂(SO4)₃.18H₂O.

5. A process according to claim 2, characterized in that the natural substrate comprises tannins (tanning agents), proteins, fatty acids, carbohydrates and resins.

6. A process according to claim 5, characterized in that the natural substrate comprises compounds derived from gallic acid like tannin.

7. A process according to claim 1, characterized in that, in step b), calcium carbonate is added as an aqueous dispersion of calcium carbonate.

8. A process according to any one of claims 1 to 7, characterized in that step a) comprises the steps:
a1)—obtaining an extract from a genipap fruit by grinding the fruit under high rotation, separating the extract from the fruit, followed by cooling and filtering;
a2)—semi-purifying the raw extract and qualifying non-dyeing compounds;
a3)—standardizing the extract by adding primary amino acids and heating the filtrate obtained up to temperatures in the range of about 50 to about 95° C., for a time that may range from 30 minutes to about 0.5 hours.

9. A process according to any one of claims 1 to 8, characterized in that the step b) comprises the steps:
b1) adding to the extract obtained in a) an aqueous suspension of calcium carbonate in approximately 2 to approximately 40%, preferably approximately 9%, by weight based on the total weight of the extract, the mixture of extract and calcium carbonate solution being kept under heating and stirring for about 30 minutes to about 5 hours, at a temperature of about 50 to about 95° C.;
b2) subsequently adding a mordent comprising a tannin solution, the mixture of extract and tannin solution being kept under heating and stirring for about 30 minutes to about 5 hours, at a temperature of about 50 to about 95° C.;
b3) filtering the solid substance obtained, washing, reducing the conductivity and drying at a temperature of about 30 to about 80° C., until the final moisture reaches about 0 to about 10%.

10. A process according to claim 9, characterized in that the aqueous solution of calcium carbonate is previously acidified with acetic acid, until reaching a pH range of about 2 to about 6.

11. A process according to any one of claims 1 to 10, characterized in that the resulting substance obtained from the precipitation of the genipap extract is insoluble in polar or non-polar substances.

12. Substances from genipap extract precipitates, characterized in that they are obtainable by the process as defined in any one of claims 1 to 11.

13. A cosmetic, pharmaceutical or food composition, characterized by comprising substances from genipap extract precipitates as defined in claim 12 and a physiologically acceptable carrier or excipient.

14. Use of substances from genipap extract precipitates as defined in claim 12, characterized in that it is in the preparation of cosmetic, pharmaceutical or food compositions.

15. A method for non-therapeutic treatment of humans or animals, characterized by comprising the topical administration of substances from genipap extract precipitates as defined in claim 12 or a cosmetic, pharmaceutical or food composition as defined in claim 13 to humans or animals.
